(19) United States
(12) Patent Application Publication
(10) Pub. No.: US 2004/0156782 A1
(43) Pub. Date: Aug. 12, 2004

(54) Methods of using indocyanine green (ICG) dye

(75) Inventors: Abu Alam, Lake Forest, IL (US); Ashok J. Chavan, Bloomington, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Akorn, Inc., Buffalo Grove, IL

(21) Appl. No.: 10/365,028

(22) Filed: Feb. 12, 2003

(51) Int. Cl. A61K 49/00

(52) U.S. Cl. 424/9.6

(57) ABSTRACT

Methods of using indocyanine green (ICG) dye for obtaining angiographic images of tissue in a patient, determining cardiac output or hepatic function and liver blood flow, and diagnosing and treating a lesion. In each method, an aqueous ICG composition is administered to the patient more than 10 hours after the ICG has been reconstituted to form the aqueous ICG composition, and up to a maximum time limit as determined using HPLC analysis of ICG.

### FIELD OF THE INVENTION

The present invention is generally directed to methods of using indocyanine green (ICG) dye.

### BACKGROUND OF THE INVENTION

Indocyanine green (ICG) is a well-known tricarbocyanine fluorescent dye. ICG is presently marketed by Akorn, Inc. (Buffalo Grove, Ill.) under the trademark IC-GREEN™ in single vials of lyophilized ICG for reconstitution with sterile Water For Injection (WFI).

The U.S. Food and Drug Administration (FDA) has approved the use of IC-GREEN™ in connection with three diagnostic procedures: cardiac output, hepatic function and liver blood flow, and ophthalmic angiography. The IC-GREEM™ may be reconstituted with WFI to provide a 5 mg ICG/ml solution in these procedures, although a 20 mg/ml aqueous solution has been found to give optimal angiograms.

The use of reconstituted ICG is limited, however. The FDA-approved package insert advises that IC-GREEN™ is unstable in aqueous solution and must be used within 10 hours after reconstitution with WFI.

A need exists for improved methods of completing FDA-approved and other diagnostic and therapeutic methods in which the use of reconstituted ICG is required or desirable.

### SUMMARY OF THE INVENTION

The present invention meets the foregoing and other needs by providing, in a first aspect, a method for obtaining an angiographic image of tissue in a patient. This method comprises: reconstituting lyophilized indocyanine green (ICG) with an aqueous diluent to provide an aqueous ICG composition; parenterally administering the aqueous ICG composition to a patient; applying energy of a type and in an amount sufficient to cause ICG in the tissue to fluoresce; obtaining an angiographic image of tissue while the ICG fluoresces, wherein the aqueous ICG composition is parenterally administered to the patient after 10 hours and while the ICG remains stable in the reconstituted aqueous composition.

Another aspect of the present invention provides a method for determining cardiac output using ICG. This method comprises: reconstituting lyophilized indocyanine green (ICG) with an aqueous diluent to provide an aqueous ICG composition; parenterally administering a known amount of the aqueous ICG composition to a patient; continuously measuring the concentration of ICG in the patient's bloodstream to determine the change in ICG concentration over time; and analyzing the ICG concentration over time to determine the patient's cardiac output, wherein the aqueous ICG composition is administered to the patient after 10 hours and while the ICG remains stable in the aqueous reconstituted composition.

A third aspect of the present invention provides a method of determining hepatic function and liver blood flow, again using ICG. This method comprises: reconstituting lyophilized indocyanine green (ICG) with an aqueous diluent to provide an aqueous ICG composition; parenterally administering a known amount of the aqueous ICG composition to a patient; measuring the concentration of ICG in the patient's bloodstream to determine the change in ICG concentration over time; and analyzing the change in ICG concentration over time to determine the rate at which the patient's liver eliminates ICG from the blood, wherein the aqueous ICG composition is parenterally administered to the patient after 10 hours and while the ICG remains stable in the aqueous reconstituted composition.

A fourth aspect of the present invention provides methods for the diagnosis and/or treatment of lesions in an animal (e.g., CNV, tumor, or other mass that includes blood vessels that feed blood into the lesion) using ICG.

In each of the foregoing methods, an aqueous ICG composition is administered to the patient more than 10 hours after reconstitution of the ICG and while the ICG remains stable in the reconstituted composition, with the desired diagnostic and/or therapeutic procedure being practiced on the animal thereafter.

These and other features and advantages of the present invention will become apparent upon review of the following detailed description of the preferred embodiments of the present invention.

### BRIEF DESCRIPTION OF THE DRAWING

FIG. 1 is a graph showing the x-ray diffraction characteristics of crystalline ICG suitable for use in the disclosed methods.

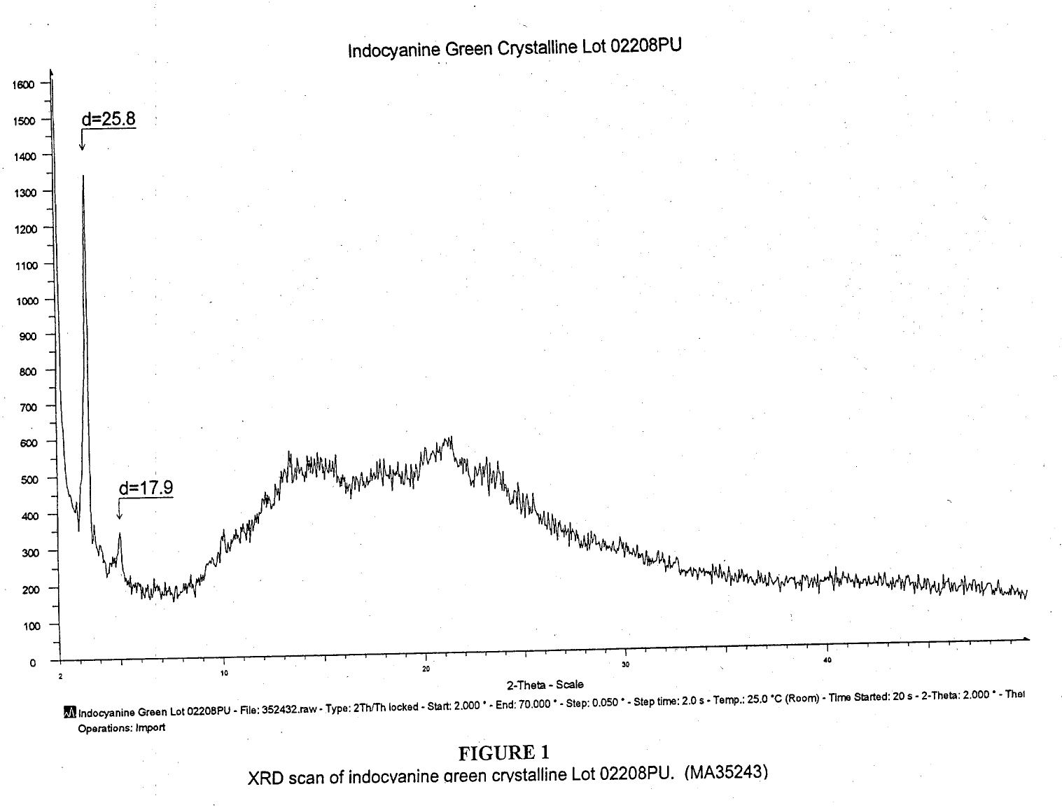

FIG. 2 is a graph showing the x-ray diffraction characteristics of lyophilized ICG suitable for use in the disclosed methods.

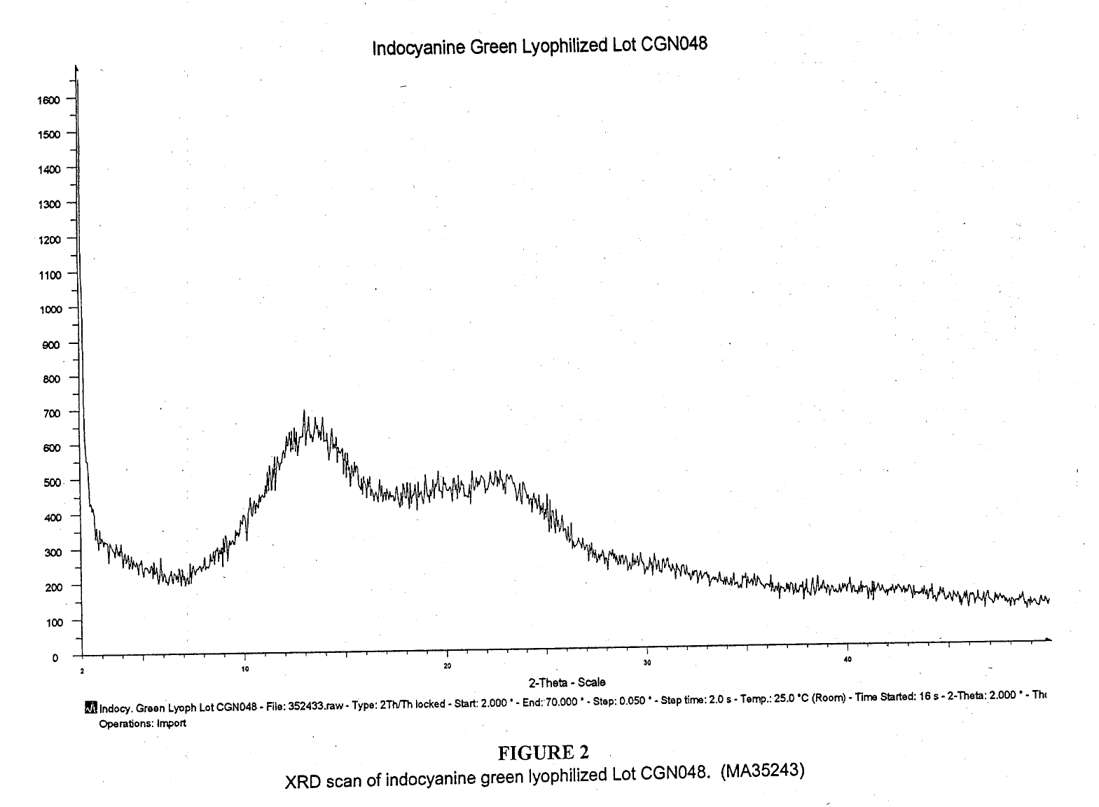

FIG. 3 provides data obtained using high pressure liquid chromatography (HPLC) for a 5 mg/ml reconstituted aqueous ICG composition (of lyophilized ICG using WFI as the diluent) indicating the loss of ICG due to degradation over time (under ambient conditions) as a percent of original label claim (i.e., 5 mg).

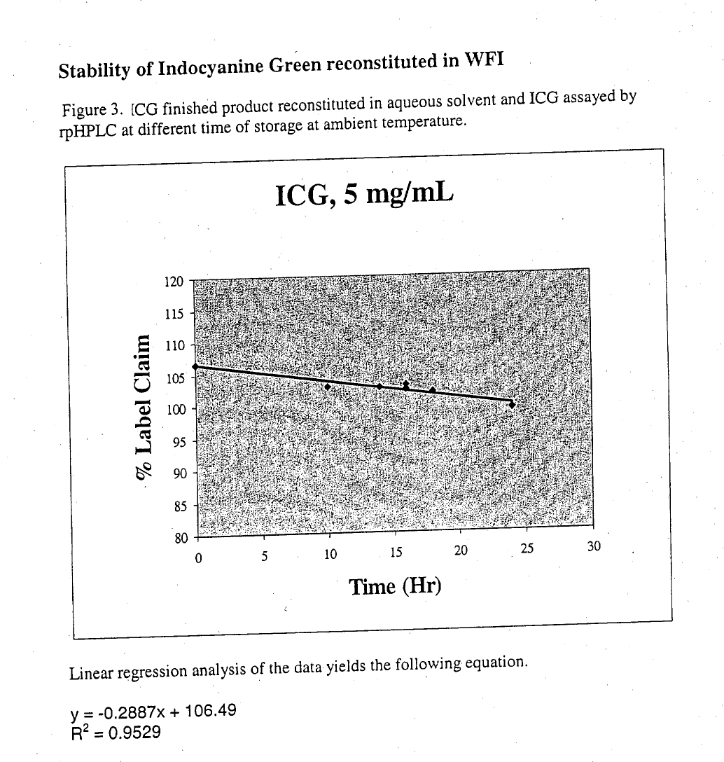

### DETAILED DESCRIPTION OF THE INVENTION

The present invention is directed to methods of using lyophilized ICG compositions that have been reconstituted with an aqueous composition, such as Water For Injection (WFI).

In a first aspect, the present invention provides a method for obtaining an angiographic image of tissue in a patient. This method comprises: reconstituting lyophilized indocyanine green (ICG) with an aqueous diluent to provide an aqueous ICG composition; parenterally administering the aqueous ICG composition to a patient; applying energy of a type and in an amount sufficient to cause ICG in the tissue to fluoresce; and obtaining an angiographic image of tissue while the ICG fluoresces, wherein the aqueous ICG composition is parenterally administered to the patient after 10 hours and while the ICG remains stable in the aqueous reconstituted composition.

Another aspect of the present invention provides a method for determining cardiac output using ICG. This method comprises: reconstituting lyophilized indocyanine green (ICG) with an aqueous diluent to provide an aqueous ICG composition; parenterally administering a known amount of the aqueous ICG composition to a patient; continuously measuring the concentration of ICG in the patient's bloodstream to determine the change in ICG concentration over time; and analyzing the ICG concentration data obtained in the prior step to determine the patient's cardiac output, wherein the aqueous ICG composition is administered to the patient after 10 hours and while the ICG remains stable in the aqueous reconstituted composition.

A third aspect of the present invention provides a method of determining hepatic function and liver blood flow, again using ICG. This method comprises: reconstituting lyophilized indocyanine green (ICG) with an aqueous diluent to provide an aqueous ICG composition; parenterally administering a known amount of the aqueous ICG composition to a patient; measuring the concentration of ICG in the patient's bloodstream to determine the change in ICG concentration over time; and analyzing the change in ICG concentration over time to determine the rate at which the patient's liver eliminates ICG from the blood, wherein the aqueous ICG composition is parenterally administered to the patient after 10 hours and while the ICG remains stable in the aqueous reconstituted composition.

Generally, the aqueous reconstituted ICG composition is administered promptly after reconstitution is completed (within about 5 minutes), but may be administered prior to a loss of ICG stability in the aqueous reconstituted composition.

The inventive methods are premised in part on the discovery that HPLC provides a means of acquiring useful qualitative and quantitative data relating to the degradation of reconstituted ICG, and the further recognition that this particular means of data acquisition is superior to known methodologies.

Using HPLC, it was further found that reconstituted ICG could be used in the aforementioned FDA-approved indications beyond the 10-hour limit presently imposed by the FDA, e.g., up to about 37 hours for a 5 mg/ml dilution, before an undesirable level of ICG degradation is realized (at ambient temperature, 25° C). The use of HPLC also assisted in the discovery that aqueous compositions having relatively high ICG concentrations are more stable relative to aqueous compositions having relatively low ICG concentrations.

Stability may be reflected in the amount of ICG that remains in a reconstituted composition per unit time. For example, a stable reconstituted ICG composition advantageously retains an ICG concentration of at least 90 wt. %, preferably at least about 95 wt. %, more preferably at least about 98 wt. %, based on the label claim.

Based upon an HPLC analysis, a 5 mg/ml reconstituted aqueous ICG composition may be used up to about 37 hours after reconstitution (corresponding to a 90 wt. % ICG content based on the label claim), or up to about 18 hours (corresponding to a 95 wt. % ICG content based on the label claim).

Based on the foregoing, it was found that the reconstituted compositions (at concentrations of ranging from about 5 mg/ml to about 20 mg/ml) may be safely administered in connection with the methods described herein more than 10 hours after reconstitution (e.g., after about 10.5 hours, after about 11 hours, or after about 12 hours).

The particular method used to measure the relative ICG concentration in the aqueous ICG composition can be any method suitable for HPLC analysis of aqueous ICG solutions.

The ICG suitable for use in the methods described herein may be provided in any suitable form, but is most commonly provided as a sterile lyophilizate. When provided as a lyophilizate, the ICG may be reconstituted prior to administration by use of water, but is advantageously reconstituted with an aqueous diluent composition, as described herein. To effect the reconstitution, water, desirably in the form of sterile WFI, may be introduced into a vial holding the ICG, with a diluent (comprising one or more components) being added thereafter, the WFI and diluent together constituting the aqueous diluent composition. Alternatively, the WFI and diluent may be added in reverse order. Preferably, however, the diluent and water are combined in a vial separate from the ICG vial, with the diluent being introduced into the ICG-containing vial in an amount sufficient to provide the desired final ICG concentration, more preferably without the need for additional dilution to obtain the desired final ICG concentration.

Preferably, the lyophilized ICG used in the inventive methods is an amorphous polymorph having a melting point of 152.1° C. (onset), a peak melting point of 153.2° C., and a decomposition peak at about 246° C., when analyzed by differential scanning calorimetry.

Iodide is also typically present in the ICG itself as a remnant of ICG synthesis. When present, iodide is desirably limited to no more than 5 wt. % (based on weight of ICG). Advantageously, the iodide content is less than about 1 wt. %, and is preferably iodide-free (as tested per USP standard).

The water included in the inventive composition is preferably sterilized, e.g., WFI. If included in the diluent, the amount of water therein should be that required to provide the desired ICG concentration in the reconstituted aqueous ICG compositions, as well as the desired weight percentages of the other diluent components.

In each of the methods described herein, the aqueous ICG composition may be parenterally administered to the patient by any means known to those of skill in the art. For example, the aqueous ICG composition may be administered via intravenous (IV) injection. With respect to the methods of obtaining angiographic images disclosed herein, the aqueous ICG composition is preferably parenterally administered to the patient in close proximity to the tissue to be examined, thereby minimizing the amount of dilution that may occur and the amount of the aqueous ICG composition that must be administered to obtain a satisfactory angiographic image.

It is well known that the peak absorption and emission of ICG lies in the range of 800-850 nm. Thus, an energy source emitting such wavelength should be used when obtaining angiographic images during a diagnostic procedure, as well as during any therapeutic procedure (with power being modulated accordingly).

The procedures associated with the three FDA-approved procedures identified herein are well known to those skilled in the art, and will not be described in detail herein.

To the extent the ICG is used in the treatment of a lesion, e.g., administration of ICG followed by irradiation of the ICG in the lesion itself or in the vessels that feed (supply) blood to the lesion, the radiation may be supplied by any suitable device. Various devices, such as fundus cameras, can be adapted for providing an appropriate level and type of energy in accordance with the teachings provided herein. The latter include, for example, those described in U.S. Pat. Nos. 5,279,298, 5,394,199 and 5,400,791. Preferably, a fundus camera having two sources of radiation (e.g., lasers) is provided. Using this type of device, one laser can be used to irradiate the general area of interest so any tissue or vessel requiring treatment can be identified (a diagnostic procedure), while the second laser can be used almost immediately upon identification of the tissue or vessel to be treated to hasten the coagulation of the blood therein, i.e., dye-enhanced photocoagulation (a therapeutic procedure). The ability to aim the treatment laser using the identical view used to obtain the angiograms is a significant advantage. Further, the ability to complete the diagnosis and treatment steps within minutes, e.g., advantageously in less than about 30 and preferably less than about 15 minutes, lessens patient trauma and increases overall treatment efficiency.

An endoscope may also be used to obtain the angiograms in each of the described methods. The endoscope would be inserted into the body and positioned adjacent the area of interest. A first instrument would be used with the endoscope to provide energy at an appropriate wavelength, e.g., a laser optic cable, to cause the ICG dye within the subject tissue to fluoresce so an angiogram can be obtained. Similarly, a second instrument would be used with the endoscope that would permit an angiographic image of the fluorescing ICG dye within the tissue to be obtained. For example, an optical device connected to a CCD camera, such as those used to perform a colonoscopy and other invasive procedures that permit a physician to view the interior of a body cavity, presently exists, and such technology may be readily adapted for use in conjunction with the endoscopic procedures of the present invention.

The inventive methods directed to obtaining angiographic images further contemplate the administration of ICG in order to permit visualization of tissue at a variety of locations within the patient's body, typically using an endoscope in the previously-described manner. Generally, angiograms of blood vessels and other abnormalities associated with blood vessels may be obtained at any location in an animal in which readable angiographic images can be obtained. For example, hollow organs and body cavities may be subjected to the inventive methods, e.g., the interior wall of the bladder, lung, gastrointestinal tract, bladder, pancreas, gall bladder, sinus, liver, kidney, heart, cervix, ovary, prostate, stomach, trachea, skin or colon may be explored, as well as the exterior walls of those organs, and the brain. This permits the diagnosis (including, e.g., the monitoring of prior treatments or of prior-diagnosed conditions) and treatment of lesions, e.g., abnormal blood vessels, such as aneurysms, ruptured blood vessels, choroidal neovascularization (CNV), as well as the diagnosis and treatment of tumors associated with those and other body cavity tissues. The treatment of tumors may be provided by the direct irradiation of the tumor itself while ICG is present therein, or by the irradiation of blood vessesl that feed blood into the tumor, thereby causing a reduction or cessation of blood flow into the tumor.

The concentration of ICG in the aqueous ICG compositions used in the inventive methods can be any concentration that is known to produce angiographic images of satisfactory quality. In a preferred embodiment, the ICG is present in the aqueous ICG composition at a concentration of at least about 5 mg/ml, more preferably at least about 10 mg/ml, most preferably at least about 20 mg/ml.

The present invention also provides methods for determining cardiac output in an animal. Generally, to determine an animal's cardiac output, a single bolus of a dye is introduced into the animal's circulatory system. Then, the concentration of the dye is continuously monitored near the point of bolus introduction. This concentration data is then used to generate a concentration/blood-level versus time curve, which is used to determine the amount of time required for the bolus to complete one cycle through the animal's circulatory system. The concentration data and time measurements are used to determine the animal's cardiac output. More specifically, the inventive methods comprise the steps of reconstituting lyophilized ICG with an aqueous diluent to provide an aqueous ICG composition, parenterally administering the aqueous ICG composition to a patient, continuously measuring the concentration of ICG in the patient's bloodstream to determine the change in ICG concentration over time, and analyzing the ICG concentration data to determine the patient's cardiac output.

In each of the methods for determining cardiac output described herein, the aqueous ICG composition may be parenterally administered to the patient by any means known to those of skill in the art. Generally, the aqueous ICG composition is rapidly administered in a single bolus to ensure that dilution of the aqueous ICG composition from the injection is minimized.

To determine the concentration of ICG in the patient's bloodstream, any method known to yield reliable measurements of ICG concentration in whole blood may be used. Generally, the concentration of ICG in the patient's bloodstream is measured spectrophotometrically. It is well known within the art that the absorption spectrum of ICG in whole blood peaks at approximately 805-810 nm. Thus, the concentration of ICG in the patient's bloodstream can be determined by measuring the amount of light absorbed by the patient's blood in the near-infrared region. In a preferred aspect, the concentration of ICG in the patient's bloodstream is measured by continuous withdrawal of arterial blood. In this embodiment, the absorption spectrum of the blood is then spectrophotometrically measured and compared to standards containing known amounts of ICG to determine the concentration of ICG in the patient's bloodstream. In another preferred aspect, the concentration of ICG in the patient's bloodstream is measured using an ear densitometer, such as that supplied by The Waters Company, Rochester, Minn., which is capable of measuring the absorption spectrum of blood in vivo.

In each of the described methods for determining cardiac output, the concentration of ICG in the aqueous ICG composition can be any concentration that permits the reliable measurement of cardiac output. In a preferred aspect of the inventive methods, the ICG is present in the aqueous ICG composition at a concentration of at least about 5 mg/ml, more preferably at least about 10 mg/ml, and most preferably at least about 20 mg/ml.

Generally, when used to measure cardiac output, the total amount of ICG administered to the patient will vary depending upon the patient's size and total blood volume. Those of ordinary skill in the art may readily determine the necessary amount using well-known methods. In a preferred aspect, the total amount of ICG administered to the patient is at least about 1 mg, more preferably at least about 2 mg, and most preferably at least about 5 mg. In another preferred aspect, the total amount ICG administered to the patient is less than about 2 mg/kg.

The present invention further provides methods for determining hepatic function and liver blood flow in an animal. It is well known to those of skill in the art that ICG is rapidly bound to plasma protein, of which albumin is the principle carrier. Generally, the bound ICG is only removed from a patient's bloodstream by the hepatic parenchymal cells. Therefore, a patient's relative hepatic function and blood flow can be determined by measuring the rate at which ICG is eliminated from the patient's bloodstream and comparing that rate to data for persons exhibiting normal hepatic function and liver blood flow. The method usually entails injecting a known amount of ICG into the vein of a patient's arm. Then, at regular intervals (e.g., every 5 minutes) or after the lapse of a predetermined amount of time (e.g., 20 minutes), the concentration of ICG in the patient's bloodstream is measured in a location far removed from the point at which the ICG was administered (e.g., a vein in the opposite arm, or the patient's ear). In particular, the inventive methods comprise the steps of reconstituting lyophilized ICG with an aqueous diluent to provide an aqueous ICG composition, parenterally administering a known amount of the aqueous ICG composition to a patient, measuring the concentration of ICG in the patient's bloodstream to determine the change in ICG concentration over time, and analyzing the ICG concentration data to determine the rate at which the patient's liver eliminates ICG from the blood.

The concentration of ICG in the patient's bloodstream can be measured using any of the methods described in connection with the inventive methods for determining cardiac output.

In a preferred aspect of the inventive methods for determining hepatic function and liver blood flow, the percentage disappearance rate (PDR) is determined by measuring the ICG concentration in the patient's bloodstream at regular intervals (e.g., 5, 10, 15, and 20 minutes after injection of the bolus). This concentration data is then plotted (e.g., using a semilogarithmic scale) and the PDR is calculated. In another preferred aspect, the percentage retention is determined by measuring the ICG concentration in the patient's bloodstream at a specified time (e.g., 20 minutes after injection of the bolus), then comparing that measurement with the known amount of ICG administered to the patient to determine the percentage of ICG retained in the patient's bloodstream.

It should be understood that in connection with the disclosed methods for determining hepatic function and liver blood flow, the concentration of ICG in the aqueous ICG composition should be sufficient to permit reliable measurement of ICG concentration in whole blood. Those skilled in the art can readily determine the amount necessary to yield such reliable measurements using well-known methods. In preferred aspects of the inventive methods, the ICG is present in the aqueous ICG composition at a concentration of at least about 5 mg/ml, more preferably at least about 10 mg/ml, and most preferably at least about 20 mg/ml.

In determining hepatic function and liver blood flow, the total amount of ICG administered to the patient will vary depending upon the physical characteristics of the particular patient (e.g., size and overall blood volume), but should be sufficient to yield an ICG concentration that permits spectrophotometic measurement of the ICG concentration in the patient's blood; however, the amount should not be so high as to cause adverse effects in the patient. In a preferred aspect of the inventive methods, the total amount of ICG administered to the patient is at least about 0.1 mg/kg of body weight, more preferably at least about 0.5 mg/kg of body weight, most preferably about 5 mg/kg of body weight.

The present invention also provides methods for diagnosing and treating a lesion in an animal, wherein a blood vessel feeds blood into the lesion. As utilized herein, the term “lesion” means any abnormal change in structure of an organ or part due to injury or disease. Lesions which may be treated using the methods described herein include, but are not limited to, ruptured blood vessels, abnormal vasculature, choroidal neovascularization (CNV), and tumors. Generally, in this method, radiation of a certain wavelength (based upon the dye used) is applied onto an undesired portion of a dye-carrying blood vessel, e.g., a vessel that carries, or feeds, blood to the lesion. The radiation, once a wavelength is applied that will “excite” the dye, causes the temperature of the dye to increase upon absorption of the radiation. While not desiring to be bound to any particular theory, as the dye temperature increases, the temperature of the surrounding blood and vessel tissue also increases. This increase in temperature hastens the rate at which blood clots in and adjacent that portion of the vessel onto which the radiation is applied. This clotting, in turn, leads to partial, or preferably complete, obstruction of the vessel in or adjacent the portion of the vessel onto which the radiation was applied. This obstruction will, in many instances, provide for subsequent reduction in the lesion. Alternatively, or in connection with this therapy, the lesion itself may be irradiated in the presence of the dye.

In particular, the inventive methods comprise the steps of reconstituting lyophilized indocyanine green (ICG) with an aqueous diluent to provide an aqueous ICG composition, parenterally administering the aqueous ICG composition to a patient, applying energy of a type and in an amount sufficient to cause the ICG to fluoresce as the ICG flows through the blood vessels, obtaining an angiographic image of the fluorescing ICG dye as the dye flows through the blood vessels, analyzing the angiographic image to determine the presence of a lesion, and applying energy to the lesion to impair the lesion and/or to the blood vessel feeding blood into the lesion of a type and in an amount sufficient to reduce the rate of rate of blood flow through the blood vessel. Impairment of the lesion can be observed both qualitatively and quantitatively via ultrasound, with a reduction in rate of growth or size being indicative of lesion impairment.

It should be appreciated that in connection with the disclosed methods for diagnosing and treating a lesion in an animal, the amount of ICG administered to the patient should be sufficient to permit the dye to fluoresce when radiation at the appropriate wavelength is applied, thereby providing useful angiographic images. The same standard is applicable to the therapeutic methods; sufficient dye should be utilized to enable the desired treatment. This information may be readily determined by those skilled in the art, and should be at least that concentration currently accepted for use in ophthalmic angiography, e.g., for diagnosis, 2 ml of a 20 mg/ml ICG solution (IC-GREEN™). Optionally, the ICG is present in the aqueous ICG composition at a concentration of at least about 5 mg/ml, preferably at least about 10 mg/ml, and most preferably at least about 20 mg/ml.

Treatment is preferably effected by applying radiation upstream of the lesion, e.g., upstream of the ruptured blood vessel, the vessel feeding the tumor, or adjacent and upstream of the abnormal blood vessels, after administration of the dye composition. The radiation is desirably applied as the dye bolus first enters the vessel to be treated, whereby the flow of blood through the vessel is reduced. Permitting the ICG to circulate within the body permits the ICG to stain the walls of those tissues that are contacted by the ICG. This may result in undesired portions of the tissue being treated. While not desiring to be bound to any particular theory, when radiation is applied, the temperature of any liquid adjacent the ICG dye receiving the radiation is raised, and the blood clotting is hastened, thereby reducing, e.g., partially or completely preventing, the flow of blood through the vessel. Varicose veins may also be treated using the aforementioned treatment methods.

When the treatment of a tumor, advantageously a solid tumor, is undertaken, the method of the present invention is preferably used in combination with other treatment agents. For example, therapeutically-effective amounts of chemotherapeutic agents, such as cisplatin, carboplatin, doxorubicin, paclitaxel, taxotere, methotrexate, fluorouracil, camptothecin, cyclophosphamide and mixtures thereof, may be administered, as well as therapeutically-effective amounts of anti-angiogenesis agents, either alone or in combination, may be administered. The identity of suitable anti-tumor and anti-angiogenesis agents and associated dosage regimens are well known, and as such will not be repeated herein. The timing of administration of these agents may occur at any time so long as the administration does not interfere with the treatment method of the present invention. Advantageously, however, the agents may be administered in combination with the dye-enhanced photocoagulation treatment methods described herein. For example, the agents can be administered immediately after dye-enhanced photocoagulation of tumor feeder vessels, and preferably are injected directly into the tumor. This provides several advantages including the reduction of trauma to the patient because multiple treatment agents are administered in a single procedure, the chemotherapeutic and anti-angiogenesis agents are delivered directly to the tumor thereby limiting the exposure of healthy tissue to these toxic agents (as would be the case using conventional IV administration), and conventional radiation can be narrowly focused on the tumor itself, as opposed to conventional methods that irradiate an area surrounding the tumor.

Conventional radiation treatment, mentioned previously, surgical intervention, and photodynamic therapy may be used individually or in combination, before, after and in some cases, if feasible, during, the diagnostic and/or treatment methods of the present invention have been used. Preferably, PDT is applied after the dye-enhanced photocoagulation therapy described herein, and more preferably without further administration of ICG. If a need for additional ICG is indicated, however, the original and additional ICG is advantageously obtained from the same source (e.g., vial).

When diagnosis of the tumor is made in accordance with the angiogram methodology of the present invention, the location and boundaries of the tumor may be determined with a high degree of precision, without resort to the use of more harmful diagnostic procedures, e.g., X-rays. The precision provided by the present invention permits the treatment agents described previously to be more efficient because they are applied with a high degree of precision onto just the tumor itself, as compared to conventional methods, e.g., system administration of chemotherapeutic agents and application of radiation, which are applied over a more general area. This precise focus, in turn, lessens trauma to the subject by minimizing the side effects of these toxic agents.

The following examples further illustrate the invention but, of course, should not be construed as in any way limiting its scope.

### EXAMPLE 1

This example demonstrates that the relative concentration (% label claim) of ICG in an aqueous ICG composition, such as that used in each of the described inventive methods, remains above 90 wt. % using HPLC testing for more than ten hours after the lyophilized ICG is reconstituted.

The HPLC testing utilized a Supelcosil LC-18-DB, 3 μm, 4.6×150 mm HPLC column and a supelcosil LC-18-DB, 3 μm, 4.6×33 mm guard column. The mobile phase, which consists of 0.1% phosphoric acid (Solvent A) and 100% acetonitrile (Solvent B), was fed to the columns at a flow rate of 1 ml/min, with the mobile phase composition being varied as follows:

| Time | % Solvent | % Solvent |
|---|---|---|
| (minutes) | A | B |
| 0 | 90 | 10 |
| 20 | 20 | 80 |
| 25 | 20 | 80 |
| 30 | 90 | 10 |
| 35 | 90 | 10 |

An ICG standard was then prepared by dissolving approximately 5.5 mg of ICG (IC-GREEN™) in 10.0 ml of a diluent comprising acetonitrile and water in a 1:1 ratio. The ICG samples to be measured (IC-GREEN™) were then prepared by diluting each sample in the same diluent (1:1 acetonitrile to water) such that the estimated concentration of ICG in each sample was approximately 0.5 mg/ml. The dilution factor was determined by dividing the final volume of the sample by the volume of the ICG sample aliquot initially added. Then, five 10.0 μL quantities of the ICG standard were injected into the HPLC column and the mean peak area was determined. Next, a series of 10.0 μL injections of the samples was completed, and the mean peak area for each sample was determined. The ICG concentration for each sample was then calculated using the following formula:

[ICG in mg/ml]=[STD](A_{SX}/A_{STD})(DF)

where [STD] is the ICG concentration of the standard, A_{SX} is the mean peak area of the sample, A_{STD} is the mean peak area of the standard, and DF is the dilution factor. The relative concentration of ICG present in the sample (wt. % label claim) was then determined by dividing the concentration of the sample by the initial concentration upon dissolution, then multiplying the result by one hundred.

The results indicate that the 5 mg ICG/ml samples retained a 90 wt. % label claim well beyond the 10 hour FDA-imposed limit, and further indicated the value of the HPLC method as opposed to existing analytical methods, e.g., the maximum time limit for use of such samples also being determined.

The methods described herein comprise a series of diagnostic and/or treatment steps. It should be understood that these methods and associated steps may be performed in any logical order, although diagnosis will typically precede therapy. Moreover, the methods may be performed alone, or in conjunction with other diagnostic procedures and treatments administered before, during or after such methods and steps set forth herein without departing from the scope and spirit of the present invention. It is further contemplated that the term animals, as used herein, includes, but is not limited to, humans.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method for obtaining an angiographic image of tissue in a patient comprising
(a) reconstituting lyophilized indocyanine green (ICG) with an aqueous diluent to provide an aqueous ICG composition;
(b) parenterally administering the, aqueous ICG composition to a patient;
(c) applying energy of a type and in an amount sufficient to cause ICG in the patient to fluoresce; and
(d) obtaining an angiographic image of the tissue while the ICG fluoresces,
wherein the aqueous ICG composition is parenterally administered to the patient after 10 but no more than about 38 hours after reconstitution of lyophilized ICG in step (a) is completed.

2. The method according to claim 1, wherein the aqueous ICG composition is parenterally administered to the patient after about 10.5 hours but no more than about 38 hours after reconstitution of lyophilized ICG in step (a) is completed.

3. The method according to claim 1, wherein the aqueous ICG composition is parenterally administered to the patient before the ICG concentration is less than 90 wt. % of its initial concentration as determined by HPLC.

4. The method according to claim 1, wherein the tissue is the eye, lung, gastrointestinal tract, bladder, pancreas, gall bladder, sinus, trachea, liver, kidney, heart, cervix, brain, ovary, prostate, stomach or skin.

5. The method according to claim 4, wherein the tissue is the eye.

6. A diagnostic method using indocyanine green comprising
(a) reconstituting lyophilized indocyanine green (ICG) with an aqueous diluent to provide an aqueous ICG composition;
(b) parenterally administering a known amount of the aqueous ICG composition to a patient;
(c) continuously measuring the concentration of ICG in the patient's bloodstream to determine the change in ICG concentration over time; and
(d) analyzing the ICG concentration data obtained in step (c),
wherein the aqueous ICG composition is parenterally administered to the patient after 10 but no more than about 38 hours after reconstitution of lyophilized ICG in step (a) is completed.

7. The method according to claim 6, wherein the aqueous ICG composition is parenterally administered to the patient after about 10.5 hours but no more than about 38 hours after reconstitution of lyophilized ICG in step (a) is completed.

8. The method according to claim 6, wherein the aqueous ICG composition is parenterally administered to the patient before the ICG concentration is less than 90 wt. % of its initial concentration as determined by HPLC.

9. The method according to claim 6, wherein the diagnostic method is used to determine cardiac output.

10. The method according to claim 6, wherein the diagnostic method is used to determine hepatic function or liver blood flow.

11. A method for treating a lesion in an animal, wherein a blood vessel feeds blood into the lesion, comprising
(a) reconstituting lyophilized indocyanine green (ICG) with an aqueous diluent to provide an aqueous ICG composition;
(b) parenterally administering the aqueous ICG composition to a patient;
(c) applying energy to the lesion of a type and in an amount sufficient to impair the lesion,
wherein the aqueous ICG composition is parenterally administered to the patient after 10 but no more than about 38 hours after reconstitution of lyophilized ICG in step (a) is completed.

12. The method according to claim 11, wherein the aqueous ICG composition is parenterally administered to the patient after about 10.5 hours but no more than about 38 hours after reconstitution of lyophilized ICG in step (a) is completed.

13. The method according to claim 11, wherein the aqueous ICG composition is parenterally administered to the patient before the ICG concentration is less than 90 wt. % of its initial concentration as determined by HPLC.

14. The method according to claim 11, wherein the tissue is the eye, lung, gastrointestinal tract, bladder, pancreas, gall bladder, sinus, trachea, liver, kidney, heart, cervix, brain, ovary, prostate, stomach or skin.

15. The method according to claim 11, further comprising applying radiation of a type and in an amount effective to provide photodynamic therapy to the lesion.

16. The method according to claim 11, further comprising applying radiation of a type and in an amount sufficient to reduce blood flow through the vessel that feeds blood into the lesion.

17. The method according to claim 11, wherein step (c) comprises applying radiation of a type and in an amount sufficient to provide dye-enhanced photocoagulation as the dye enters the targeted tissue, and subsequently applying radiation of a type and in an amount to provide for photodynamic therapy.

18. A method for treating a tumor in an animal, wherein a blood vessel feeds blood into the tumor, comprising
(a) reconstituting lyophilized indocyanine green (ICG) with an aqueous diluent to provide an aqueous ICG composition;
(b) parenterally administering the aqueous ICG composition to a patient;
(c) applying energy to the tumor of a type and in an amount sufficient to reduce the rate of blood flow through the tumor,
wherein the aqueous ICG composition is parenterally administered to the patient after 10 but no more than about 38 hours after reconstitution of lyophilized ICG in step (a) is completed.

19. A method for treating a choroidal neovascularization (CNV) comprising
(a) reconstituting lyophilized indocyanine green (ICG) with an aqueous diluent to provide an aqueous ICG composition;
(b) parenterally administering the aqueous ICG composition to a patient;
(c) applying energy to the CNV of a type and in an amount sufficient to reduce the rate of blood flow through the CNV,
wherein the aqueous ICG composition is parenterally administered to the patient after 10 but no more than about 38 hours after reconstitution of lyophilized ICG in step (a) is completed.
